(19) United States
(12) Patent Application Publication
(10) Pub. No.: US 2024/0225966 A1
(43) Pub. Date: Jul. 11, 2024

(54) COMPOSITION FOR CARING FOR AND/OR MAKING UP KERATIN MATERIALS

(71) Applicant: L'OREAL, Paris (FR)

(72) Inventors: Shan WU, Shanghai (CN); Xiuxia WANG, Shanghai (CN)

(73) Assignee: L'OREAL, Paris (FR)

(21) Appl. No.: 18/562,927

(22) PCT Filed: Aug. 31, 2021
(86) PCT No.: PCT/CN2021/115530

§ 371 Date: Nov. 21, 2023

(51) Int. Cl. A61K 8/06; A61K 8/04; A61K 8/36; A61K 8/37; A61K 8/73; A61K 8/81; A61K 8/86; A61Q 19/00

(52) U.S. Cl. CPC A61K 8/062; A61K 8/042; A61K 8/361; A61K 8/375; A61K 8/732; A61K 8/8147; A61K 8/86; A61Q 19/001; A61K 2800/41

(57) ABSTRACT

A composition in the form of an oil-in-water emulsion for caring for and/or making up keratin materials, comprises: (i) at least one structuring agent selected from saturated C14-C22 fatty acids; (ii) at least one hydrophilic gelling agent capable of swelling in water to form a microgel in the form of particles with a particle size of 50-400 μm; and (iii) at least one surfactant. A non-therapeutic method for caring for and/or making up keratin materials, comprises applying said composition to the skin.

### TECHNICAL FIELD

The present invention relates to a cosmetic composition. In particular, the present invention relates to a composition for caring for and/or making up keratin materials. The present invention also relates to a non-therapeutic method for caring for and/or making up keratin materials.

### BACKGROUND ART

The skin is the protective barrier for the human body. It protects the interior of the body from physical injury (such as trauma) and biological injury (such as bacteria, viruses or fungi).

The development of formulations dedicated to caring for and/or making up the skin and/or lips, is permanent.

For example, efforts have been made to formulate compositions in the form of creams or emulsions. Thanks to a suitable consistency of the compositions, it is possible to provide to the skin moisturizing feeling via occlusion.

However, these types of products are not satisfying. Due to a relatively high consistency of the products, it is not easy to apply on the skin evenly.

There is thus still a need to formulate a composition for caring for the skin, which has a suitable consistency and can be easily spread on the skin during application.

### SUMMARY OF THE INVENTION

The inventors have now discovered that it is possible to formulate such compositions having the desired properties as described above.

Accordingly, in a first aspect, the present invention provides a composition in the form of an oil-in-water emulsion for caring for and/or making up keratin materials, comprising:
(i) at least one structuring agent selected from saturated C14-C22 fatty acids;
(ii) at least one hydrophilic gelling agent capable of swelling in water to form a microgel in the form of particles with a particle size of 50-400 μm; and
(iii) at least one surfactant.

The composition of the present invention is in the form of an oil-in-water emulsion. Thus, said composition comprises a continuous aqueous phase and a dispersed fatty phase.

The composition of the present invention has a suitable consistency.

The inventors found that the composition of the present invention has a lamellar-microgel inter-penetration network (IPN) texture, which can provide a good spreadability and a non-sticky skin feeling.

In a second aspect, the present invention provides a non-therapeutic method for caring for and/or making up keratin materials, comprising applying the composition according to the first aspect of the present invention to the keratin materials.

Other advantages of the present invention will emerge more clearly on reading the description and the examples that follow.

### BRIEF DESCRIPTION OF THE DRAWINGS

Implementations of the present invention will now be described, by way of example only, with reference to the attached figure, wherein:

FIG. 1 shows polarized light microscopy image of the composition obtained in invention example 1.

### DETAILED DESCRIPTION OF THE INVENTION

Unless otherwise defined, all technical and scientific terms used herein have the same meaning as commonly understood by those skilled in the art the present invention belongs to. When the definition of a term in the present description conflicts with the meaning as commonly understood by those skilled in the art the present invention belongs to, the definition described herein shall apply.

In that which follows and unless otherwise indicated, the limits of a range of values are included within this range, in particular in the expressions “between . . . and . . . ” and “from . . . to . . . ”.

Moreover, the expression “at least one” used in the present description is equivalent to the expression “one or more”.

Throughout the instant application, the term “comprising” is to be interpreted as encompassing all specifically mentioned features as well optional, additional, unspecified ones. As used herein, the use of the term “comprising” also discloses the embodiment wherein no features other than the specifically mentioned features are present (i.e. “consisting of”).

Unless otherwise specified, all numerical values expressing amount of ingredients and the like which are used in the description and claims are to be understood as being modified by the term “about”. Accordingly, unless indicated to the contrary, the numerical values and parameters described herein are approximate values which are capable of being changed according to the desired purpose as required.

For the purposes of the present invention, the term “keratin materials” is intended to cover human skin, mucous membranes such as the lips. Facial skin is most particularly considered according to the present invention.

All percentages in the present invention refer to weight percentage, unless otherwise specified.

According to the first aspect, the present invention provides a composition in the form of an oil-in-water emulsion for caring for and/or making up keratin materials, comprising:
(i) at least one structuring agent selected from saturated C14-C22 fatty acids;
(ii) at least one hydrophilic gelling agent capable of swelling in water to form a microgel in the form of particles with a particle size of 50-400 μm; and
(iii) at least one surfactant.

### Saturated C14-C22 Fatty Acids

According to the first aspect, the composition of the present invention comprises at least one at least one structuring agent selected from saturated C14-C22 fatty acids.

As examples of saturated C14-C22 fatty acid, mention can be made of myristic acid, pentadecanoic acid, palmitic acid, heptadecanoic acid, stearic acid, cetearylic acid, arachidic acid, and behenic acid.

Advantageously, the structuring agent is selected from saturated C14-C18 fatty acids, preferably, linear and saturated C14-C18 fatty acids, in particular selected from myristic acid, palmitic acid, stearic acid, and mixtures thereof.

Advantageously, the structuring agent is present in an amount ranging from 0.5 wt. % to 30 wt. %, preferably from 1 wt. % to 20 wt. %, more preferably from 1.5 wt. % to 10 wt. %, most preferably from 1.5 wt. % to 7 wt. %, relative to the total weight of the composition.

### Hydrophilic Gelling Agent

According to the first aspect, the composition according to the present invention comprises at least one hydrophilic gelling agent capable of swelling in water to form a microgel in the form of particles with a particle size of 50-400 μm.

Advantageously, the hydrophilic gelling agent is selected from alkali metal salts of acrylic acid homopolymers, modified starches, and mixtures thereof.

The homopolymer suitable for use in the composition according to the invention is chosen in particular from sodium polyacrylates and potassium polyacrylates. Sodium polyacrylate is preferably used.

The acrylic acid homopolymer may be advantageously neutralized to a degree ranging from 5% to 80%.

As sodium polyacrylate that can be used according to the invention, use may be made of those sold under the names Cosmedia SPL® by the company Cognis, or else Luvigel® EM sold by the company BASF, the product sold by the company Sensient under the trade reference Covacryl MV 60® or else under the trade name Cosmedia® by Cognis.

The modified starches suitable for use in the composition according to the invention is chosen in particular from those derived from maize starch, rice starch, cassava starch, potato starch, wheat starch, sorghum starch, pea starch, and mixtures thereof.

Among modified starches, mention may be made of precooked starches, hydrolysed starches, crosslinked starches, for example crosslinked with a methylolurea derivative or with octenylsuccinic anhydride or else with epichlorohydrin, esterified starches, etherified starches, oxidized starches, refined starches, starches roasted in the presence of an acid, or else grafted starches, for example grafted with sodium polyacrylates, coated starches, for example coated with amino acids, and/or mixtures thereof.

Among the modified starches that are particularly suitable for the invention, mention may be made of:
that sold under the names Structure XL and Structure Zea (hydroxypropyl starch phosphate) by the company National Starch;
the starches sold under the names Sanfresh ST-100C, ST100MC, IM-300MC (INCI name sodium polyacrylate starch) by the company Sanyo Chemical Industries; and
mixtures thereof.

Preferably, the hydrophilic gelling agent is selected from sodium polyacrylates, potassium polyacrylates, sodium polyacrylate starch, sodium polyacrylate starch, and mixtures thereof.

Advantageously, the hydrophilic gelling agent is present in an amount ranging from 0.1 wt. % to 10 wt. %, preferably from 0.2 wt. % to 8 wt. %, more preferably from 0.3 wt. % to 5 wt. %, most preferably from 0.3 wt. % to 2 wt. %, relative to the total weight of the composition.

### Surfactants

According to the first aspect, the composition of the present invention comprises at least one surfactant.

Preferably, the surfactant is selected from
mono and polyglyceryl esters of a fatty acid or their ethoxylated derivatives;
esters of polyols with fatty acids with a saturated or unsaturated chain;
sorbitol esters of C8-C24 fatty acids and polyoxyalkylenated derivatives thereof;
esters of fatty acids and glucose or alkylglucose or their ethoxylated derivatives;
sucrose esters;
ethers of a sugar and of C8-C24 fatty alcohols, such as caprylyl/capryl glucoside;
C14-C24 fatty alcohols;
polyoxyethylene alkyl ether carboxylic acids, such as Laureth-5 Carboxylic Acid;
polyoxyethylenated fatty alcohol containing from 6 to 12 oxyethylene units, such as Laureth-9;
polyoxyalkylenated derivative of mono glyceryl ester of a fatty acid such as PEG-20 glyceryl triisostearate,
sarcosinates, such as sodium lauroyl sarcosinate;
fatty acid ester of polyalkylene glycol; and
mixtures thereof.

As polyglyceryl esters of (a) fatty acid(s), mention be made of the product containing 2 to 10 glycerol units, such as polyglyceryl monolaurate, oleate, myristate, caprylate, or stearate comprising 2 to 10 glycerol units, polyglyceryl mono(iso)stearate comprising 2 to 10 glycerol units, polyglyceryl dioleate comprising 2 to 10 glycerol units, polyglyceryl dilaurate comprising 2 to 10 glycerol units, polyglyceryl dimyristate comprising 2 to 10 glycerol units, polyglyceryl trimyristate comprising 2 to 10 glycerol units, polyglyceryl trioleate comprising 2 to 10 glycerol units, and polyglyceryl tricaprylate comprising 2 to 10 glycerol units.

Polyglyceryl esters include, polyglyceryl esters of C16-C22 saturated, unsaturated and branched chain fatty acids, such as polyglyceryl-4 isostearate, polyglyceryl-3 oleate, polyglyceryl-2 oleate, polyglyceryl-2 sesquioleate, triglyceryl diisostearate, diglyceryl monooleate, tetraglyceryl monooleate, or mixtures thereof.

Monoglyceryl esters include, but are not limited to, glyceryl monoesters, preferably glyceryl monoesters of C16-C22 saturated, unsaturated and branched chain fatty acids such as glyceryl oleate, glyceryl monostearate, glyceryl monoisostearate, glyceryl monopalmitate, glyceryl monobehenate, or mixtures thereof.

As mono glyceryl esters of fatty acids, glyceryl stearate (glyceryl mono-, di- and/or tristearate) (CTFA name: glyceryl stearate) or glyceryl ricinoleate or mixtures thereof can be cited, or as polyoxyalkylenated derivatives thereof, mono-, di- or triester of fatty acids with a polyoxyalkylenated glycerol (mono-, di- or triester of fatty acids with a polyalkylene glycol ether of glycerol), preferably polyoxyethylenated glyceryl stearate (mono-, di- and/or tristearate), such as PEG-20 glyceryl stearate (mono-, di-, tristearate and/or triisostearate) can be cited. Preferably, the polyoxyalkylenated derivative of mono glyceryl ester of fatty acids includes 10 to 40 oxyethylene units, such as PEG-20 glyceryl triisostearate.

The surfactant may be selected from esters of polyols with fatty acids with a saturated or unsaturated chain containing for example from 8 to 24 carbon atoms, preferably 12 to 22 carbon atoms, or polyoxyalkylenated derivatives thereof, preferably containing from 10 to 200, or more preferably from 10 to 100 oxyalkylene units, such as mono glyceryl esters or poly glyceryl esters of a C8-C24, preferably C12-C22, fatty acid and polyoxyalkylenated derivatives thereof, preferably containing from 10 to 200, or more preferably from 10 to 100 oxyalkylene units; sorbitol esters of a C8-C24, preferably C12-C22, fatty acid or polyoxyalkylenated derivatives thereof, preferably containing from 10 to 200, or more preferably from 10 to 100 oxyalkylene units; sugar (sucrose, maltose, glucose, fructose, and/or alkylglycose) esters of a C8-C24, preferably C12-C22, fatty acid or polyoxyalkylenated derivatives thereof, preferably containing from 10 to 200, or more preferably from 10 to 100 oxyalkylene units; ethers of fatty alcohols; ethers of sugar and a C8-C24, preferably C12-C22, fatty alcohol or alcohols; or mixtures thereof.

Mixtures of these surfactants, such as for example the product containing glyceryl stearate and PEG-100 stearate, marketed under the name ARLACEL 165 by CRODA, and the product containing glyceryl stearate (glyceryl mono- and distearate) and potassium stearate marketed under the name TEGIN by Goldschmidt (CTFA name: glyceryl stearate SE), can also be used.

The sorbitol esters of C8-C24 fatty acids and polyoxyalkylenated derivatives thereof can be selected from sorbitan palmitate, sorbitan isostearate, sorbitan trioleate and esters of fatty acids and alkoxylated sorbitan containing for example from 20 to 100 EO, such as for example sorbitan monostearate (CTFA name: sorbitan stearate), sold by the company ICI under the name Span 60, sorbitan monopalmitate (CTFA name: sorbitan palmitate), sold by the company ICI under the name Span 40, or sorbitan tristearate 20 EO (CTFA name: polysorbate 65), sold by the company ICI under the name Tween 65, polyethylene sorbitan trioleate (polysorbate 85) or the compounds marketed under the trade names Tween 20 or Tween 60 by Uniqema.

As esters of fatty acids and glucose or alkylglucose, glucose palmitate, alkylglucose sesquistearates such as methylglucose sesquistearate, alkylglucose palmitates such as methylglucose or ethylglucose palmitate, methylglucoside fatty esters, the diester of methylglucoside and oleic acid (CTFA name: Methyl glucose dioleate), the mixed ester of methylglucoside and the mixture of oleic acid/hydroxystearic acid (CTFA name: Methyl glucose dioleate/hydroxystearate), the ester of methylglucoside and isostearic acid (CTFA name: Methyl glucose isostearate), the ester of methylglucoside and lauric acid (CTFA name: Methyl glucose laurate), the mixture of monoester and diester of methylglucoside and isostearic acid (CTFA name: Methyl glucose sesqui-isostearate), the mixture of monoester and diester of methylglucoside and stearic acid (CTFA name: Methyl glucose sesquistearate) and in particular the product marketed under the name Glucate SS by AMERCHOL, and mixtures thereof can be cited.

As ethoxylated ethers of fatty acids and glucose or alkylglucose, ethoxylated ethers of fatty acids and methylglucose, and in particular the polyethylene glycol ether of the diester of methylglucose and stearic acid with about 20 moles of ethylene oxide (CTFA name: PEG-20 methyl glucose distearate) such as the product marketed under the name Glucam E-20 distearate by AMERCHOL, the polyethylene glycol ether of the mixture of monoester and diester of methyl-glucose and stearic acid with about 20 moles of ethylene oxide (CTFA name: PEG-20 methyl glucose sesquistearate) and in particular the product marketed under the name Glucamate SSE-20 by AMERCHOL and that marketed under the name Grillocose PSE-20 by GOLDSCHMIDT, or mixtures thereof, can for example be cited.

As sucrose esters, saccharose palmito-stearate, saccharose stearate and saccharose monolaurate can for example be cited.

As sugar ethers, alkylpolyglucosides can be used, and for example, ethers of a sugar and of C8-C24 fatty alcohols including decylglucoside such as the product marketed under the name MYDOL 10 by Kao Chemicals, the product marketed under the name PLANTAREN 2000 by Henkel, and the product marketed under the name ORAMIX NS 10 by Seppic, caprylyl/capryl glucoside such as the product marketed under the name ORAMIX CG 110 by Seppic or under the name LUTENSOL GD 70 by BASF, laurylglucoside such as the products marketed under the names PLANTAREN 1200 N and PLANTACARE 1200 by Henkel, coco-glucoside such as the product marketed under the name PLANTACARE 818/UP by Henkel, cetostearyl glucoside possibly mixed with cetostearyl alcohol, marketed for example under the name MONTANOV 68 by Seppic, under the name TEGO-CARE CG90 by Goldschmidt and under the name EMULGADE KE3302 by Henkel, arachidyl glucoside, for example in the form of the mixture of arachidyl and behenyl alcohols and arachidyl glucoside marketed under the name MONTANOV 202 by Seppic, cocoylethylglucoside, for example in the form of the mixture (35/65) with cetyl and stearyl alcohols, marketed under the name MONTANOV 82 by Seppic, or mixtures thereof can in particular be cited.

As C14-C24 fatty alcohols, mention can be made of stearyl alcohol, cetyl alcohol, behenyl alcohol, arachidyl alcohol, lignocerylic alcohol and mixtures thereof.

Preferably, the fatty acid ester of polyalkylene glycol is an ester of polyethylene glycol comprising from 1 to 200 oxyethylene groups and of a saturated or unsaturated fatty acid comprising from 12 to 30 carbon atoms, more preferably, the fatty acid ester of polyalkylene glycol is selected from polyethylene glycol 2 OE monostearate or polyethylene glycol 100 monostearate (PEG-100 stearate).

Most preferably, the surfactant is selected from mono- and polyglyceryl esters of a fatty acid, ethers of a sugar and of C8-C24 fatty alcohols, fatty acid esters of polyalkylene glycol, C14-C24 fatty alcohols, and mixtures thereof.

Advantageously, the surfactant is selected from glyceryl stearate, PEG-100 stearate, arachidyl alcohol, behenyl alcohol, arachidyl glucoside, and mixtures thereof.

Advantageously, the surfactant is present in an amount ranging from 0.2 wt. % to 20 wt. %, preferably from 0.4 wt. % to 15 wt. %, more preferably from 0.8 wt. % to 12 wt. %, relative to the total weight of the composition.

### Saponifiers

Preferably, the composition of the present invention comprises at least one saponifier.

The saponifier can be used to react with a saturated C14-C22 fatty acid to produce a salt.

The saponifier can be, for example, inorganic bases, such as alkali metal hydroxides (for example, sodium hydroxide and potassium hydroxide), alkaline earth metal hydroxides (for example, magnesium hydroxide) or ammonium hydroxide, or organic bases, for example, monoethanolamine (MEA), diethanolamine, triethanolamine, tromethamine, N-methylglucamine, arginine, lysine and arginine.

Mention can be also made of sodium carbonate, potassium carbonate, sodium bicarbonate, and potassium bicarbonate.

Preferably, the saponifier is selected from sodium hydroxide, potassium hydroxide, magnesium hydroxide, ammonium hydroxide, monoethanolamine, diethanolamine, triethanolamine, tromethamine, N-methylglucamine, arginine, lysine, arginine, and mixtures thereof.

If presents, preferably, the molar ratio of the saponifier to the structuring agent selected from saturated C14-C22 fatty acids is from 0.1 mol % to 30 mol %, advantageously, from 1 mol % to 20 mol %, more preferably from 1 mol % to 10 mol %.

### Aqueous Phase

As an oil-in-water emulsion, the cosmetic composition of the present invention comprises a continuous aqueous phase.

Said aqueous phase comprises water.

Preferably, the continuous aqueous phase comprises an organic solvent miscible with water (at room temperature 25° C.) such as glycerin and glycols having from 2 to 20 carbon atoms, preferably from 2 to 10 carbon atoms, and preferentially having from 2 to 6 carbon atoms, such as propylene glycol, butylene glycol, pentylene glycol, hexylene glycol, caprylyl glycol, dipropylene glycol, diethylene glycol; and mixtures thereof.

Advantageously, said aqueous phase is present in an amount ranging from 30% to 95% by weight, preferably from 35% to 90% by weight, and more preferably from 40% to 85% by weight of the total weight of the composition.

### Fatty Phase

As an oil-in-water emulsion, the composition of the present invention comprises at least one dispersed fatty phase.

Said fatty phase comprises at least one oil.

The oil can be volatile or non-volatile.

The term “oil” means a water-immiscible non-aqueous compound that is liquid at room temperature (25° C.) and at atmospheric pressure (760 mmHg). The term “non-volatile oil” means an oil that may remain on keratin materials at room temperature and atmospheric pressure for at least several hours and that especially has a vapour pressure of less than 10⁻³ mmHg (0.13 Pa). A non-volatile oil may also be defined as having an evaporation rate such that, under the conditions defined previously, the amount evaporated after 30 minutes is less than 0.07 mg/cm².

These oils may be of plant, mineral or synthetic origin.

Said oil can be selected from hydrocarbonated, silicone or fluorinated oils.

The term “hydrocarbon-based oil” means an oil formed essentially from, or even constituted by, carbon and hydrogen atoms, and optionally O and N atoms, and free of Si and F heteroatoms. Such oil can contain alcohol, ester, ether, carboxylic acid, amine and/or amide groups.

The term “silicone oil” means an oil containing at least one silicon atom, especially containing Si—O groups.

The term “fluorinated oil” means an oil containing at least one fluorine atom.

Preferably, the oil is selected from hydrocarbon-based oils, for example, ester oils, such as caprylic/capric triglyceride, isononyl isononanoate, pentaerythrityl tetraisostearate, myristyl myristate.

The fatty phase can also comprise a non-oil fatty substance, for example butters and waxes, such as butyrospermum parkii butter, cera alba, beeswax, so as to build texture and deliver a suitable skin finish.

Advantageously, the fatty phase is present in an amount ranging from 2% to 40% by weight, preferably from 8% to 35% by weight, more preferably from 10% to 30% by weight, relative to the total weight of the composition of the present invention.

### Cosmetic Active Ingredients

Preferably, the composition of the present invention comprises a cosmetic active ingredient.

As examples of cosmetic active ingredient, mention can be made of natural extracts; vitamins such as vitamin A (retinol), vitamin E (tocopherol), vitamin C (ascorbic acid), vitamin B5 (panthenol), vitamin B3 (niacinamide), and derivatives of said vitamins (in particular esters) and mixtures thereof; urea; caffeine; C-glycosides such as hydroxypropyl tetrahydropyrantriol; salicylic acid and derivatives thereof; alpha-hydroxyacids such as lactic acid or glycolic acid and derivatives thereof; sunscreens; extracts from algae, fungi, plants, yeasts and bacteria; enzymes; agents acting on the microcirculation, and mixtures thereof.

It is easy for the skilled in the art to adjust the amount of the additional cosmetic active ingredient based on the final use of the composition according to the present invention.

### Additional Adjuvants or Additives

The composition of the present invention may comprise may also contain conventional cosmetic adjuvants or additives, for instance fragrances, chelating agents (for example, tetrasodium glutamate diacetate and disodium EDTA), preserving agents (for example, chlorphenesin and phenoxyethanol) and bactericides, pH regulators (for example citric acid), fillers, and mixtures thereof.

The skilled in the art can select the amount of the additional adjuvants or additive so as not to adversely impact the final use of the composition according to the present invention.

According to a particularly preferred embodiment, the present invention provides a composition in the form of an oil-in-water emulsion for caring for and/or making up keratin materials comprising, relative to the total weight of the composition:
(i) from 1.5 wt. % to 7 wt. % of at least one structuring agent selected from linear and saturated C14-C18 fatty acids;
(ii) from 0.3 wt. % to 2 wt. % of at least one hydrophilic gelling agent selected from alkali metal salts of acrylic acid homopolymers, modified starches, and mixtures thereof; and
(iii) from 0.8 wt. % to 12 wt. % of at least one surfactant selected from mono- and polyglyceryl esters of a fatty acid, ethers of a sugar and of C8-C24 fatty alcohols, fatty acid esters of polyalkylene glycol, C14-C24 fatty alcohols, and mixtures thereof.

### Galenic Form and Method

The composition of the present invention is in the form of oil-in-water emulsion.

The composition according to the present invention has a lamellar-microgel inter-penetration network texture.

The term “lamellar-microgel inter-penetration network texture” means a liquid crystal structure, or a swollen or non-swollen crystalline lamellar hydrate phase with plane symmetry, comprising several amphiphilic bilayers arranged in parallel and separated by a liquid medium which is a gel.

The composition of the present invention can be, for example, cream or hair conditioner.

The composition of the present invention can be used for caring for and/or making up keratin materials, for example, it can be used as foundation cream, hand cream, body cream, and so on.

According to the second aspect, the present invention provides a non-therapeutic method for caring for and/or making up keratin materials, comprising applying the composition according to the first aspect of the present invention to the keratin materials.

The following examples serve to illustrate the present invention without, however, being limiting in nature.

### EXAMPLES

Main raw materials used, trade names and supplier thereof are listed in Table 1.

TABLE 1

| INCI Name | Trade Name | Supplier |
|---|---|---|
| GLYCERYL STEARATE (and) | ARLACEL ™ 165 | CRODA |
| PEG-100 STEARATE | | |
| SODIUM POLYACRYLATE | COVACRYL ® MV60 | SENSIENT |
| STEARIC ACID | PALMAC ® 50-18/MB | IOI GROUP |
| | | OLEOCHEMICALS |
| POTASSIUM HYDROXIDE | LESSIVE DE POTASSE | QUARON |
| | 50% | |
| ARACHIDYL ALCOHOL (and) | MONTANOV 202 | SEPPIC |
| BEHENYL ALCOHOL (and) | | |
| ARACHIDYL GLUCOSIDE | | |
| CAPRYLIC/CAPRIC | MASESTER E7000 | PT MUSIM MAS |
| TRIGLYCERIDE | LR01-1/MB | |
| CERA ALBA | CERABEIL LOR | BAERLOCHER |
| | | (CERESINE) |
| BUTYROSPERMUM PARKII | FAIR FOR LIFE | OLVEA |
| BUTTER | REFINED SHEA | |
| | BUTTER | |
| ISONONYL ISONONANOATE | ERCAREL ISN/O | ERCA |
| PENTAERYTHRITYL | JOLEE 7181 | OLEON |
| TETRAISOSTEARATE | | |
| MYRISTYL MYRISTATE | TEGOSOFT ® MM | EVONIK |
| | | GOLDSCHMIDT |
| CHLORPHENESIN | MACROCIDE-OL | MACROCARE |
| AMMONIUM | HOSTACERIN AMPS | CLARIANT |
| POLYACRYLOYLDIMETHYL | | |
| TAURATE | | |
| CARBOMER 980 | CARBOPOL ® 980 | LUBRIZOL |
| | POLYMER | |
| HYDROXYPROPYL | MEXORYL SCS | CHIMEX (NOVEAL) |
| TETRAHYDROPYRANTRIOL | | |

### Invention Examples 1-5 and Comparative Examples 1-4

Compositions of invention examples (IE.) 1-5 and comparative examples (CE.) 1-4 were prepared according to the amounts given in Table 2. The amount of each component is given in % by weight of the total weight of the composition containing it.

TABLE 2

| | IE. 1 | IE. 2 | IE. 3 | IE. 4 | IE. 5 | CE. 1 | CE. 2 | CE. 3 | CE4 |
|---|---|---|---|---|---|---|---|---|---|
| POTASSIUM HYDROXIDE | 0.02 | 0.02 | 0.12 | — | — | 0.02 | 0.02 | 0.02 | 0.02 |
| STEARIC ACID | 1.50 | 6.70 | 6.00 | 1.50 | 6.00 | 1.50 | 6.70 | 6.70 | — |
| GLYCERYL STEARATE (and) | 0.80 | 1.00 | 1.20 | 0.80 | 1.00 | 1.00 | — | 1.00 | 1.00 |
| PEG-100 STEARATE | | | | | | | | | |
| ARACHIDYL ALCOHOL (and) | 2.00 | 2.50 | 2.00 | 2.00 | 2.50 | 2.00 | 2.50 | 2.50 | 2.50 |
| BEHENYL ALCOHOL (and) | | | | | | | | | |
| ARACHIDYL GLUCOSIDE | | | | | | | | | |
| WATER | To 100 | To 100 | To 100 | To 100 | To 100 | To 100 | To 100 | To 100 | To 100 |
| GLYCERIN | 8.00 | 8.00 | 8.00 | 8.00 | 8.00 | 8.00 | 8.00 | 8.00 | 8.00 |
| CAPRYLYL GLYCOL | 0.30 | 0.30 | 0.30 | 0.30 | 0.30 | 0.30 | 0.30 | 0.30 | 0.30 |
| CAPRYLIC/CAPRIC | 2.00 | 1.00 | — | 2.00 | 1.00 | 2.00 | 1.00 | 1.00 | 1.00 |
| TRIGLYCERIDE | | | | | | | | | |
| CERA ALBA | 2.73 | 2.40 | 2.40 | 2.73 | 2.40 | 2.73 | 2.40 | 2.40 | 2.40 |
| BUTYROSPERMUM PARKII | 3.55 | 3.12 | 3.10 | 3.55 | 3.12 | 3.55 | 3.12 | 3.12 | 3.12 |
| BUTTER | | | | | | | | | |
| ISONONYL ISONONANOATE | 1.00 | 1.00 | 1.00 | 1.00 | 1.00 | 1.00 | 1.00 | 1.00 | 1.00 |
| PENTAERYTHRITYL | 1.00 | 0.80 | 0.40 | 1.00 | 0.80 | 1.00 | 0.80 | 0.80 | 0.80 |
| TETRAISOSTEARATE | | | | | | | | | |
| MYRISTYL MYRISTATE | 1.23 | 1.10 | 1.10 | 1.23 | 1.10 | 1.23 | 1.10 | 1.10 | 1.10 |
| CHLORPHENESIN | 0.20 | 0.20 | 0.20 | 0.20 | 0.20 | 0.20 | 0.20 | 0.20 | 0.20 |
| SODIUM POLYACRYLATE | 0.80 | 0.60 | 0.60 | 0.80 | 0.60 | — | 0.60 | — | 0.60 |
| AMMONIUM | — | — | — | — | — | 0.80 | — | — | — |
| POLYACRYLOYLDIMETHYL | | | | | | | | | |
| TAURATE | | | | | | | | | |
| CARBOMER 980 | — | — | — | — | — | — | — | 0.60 | — |
| HYDROXYPROPYL | 15.00 | 15.00 | 15.00 | 15.00 | 15.00 | 15.00 | 15.00 | 15.00 | 15.00 |
| TETRAHYDROPYRANTRIOL | | | | | | | | | |

Compositions of invention examples 1-5 are compositions according to the present invention.

Composition of comparative example 1 comprises ammonium polyacryloyldimethyl taurate rather than a hydrophilic gelling agent capable of swelling in water to form a microgel in the form of particles with a particle size of 50-400 μm.

Composition of comparative example 2 does not comprise a surfactant.

Composition of comparative example 3 comprises Carbomer 980 rather than a hydrophilic gelling agent capable of swelling in water to form a microgel in the form of particles with a particle size of 50-400 μm.

Composition of comparative example 4 does not comprise a structuring agent selected from saturated C14-C22 fatty acids.

### Preparation Process:

The compositions listed above were prepared as follows, taking the composition of invention example 1 as an example:

TABLE 3

| Phase | INCI name | IE. 1 |
|---|---|---|
| A1 | WATER | 59.62 |
| | CHLORPHENESIN | 0.20 |
| | CAPRYLYL GLYCOL | 0.30 |
| | GLYCERIN | 8.00 |
| A2 | HYDROXYPROPYL TETRAHYDROPYRANTRIOL | 15.00 |
| B | STEARIC ACID | 1.50 |
| | ARACHIDYL ALCOHOL (and) BEHENYL ALCOHOL | 2.00 |
| | (and) ARACHIDYL GLUCOSIDE | |
| | GLYCERYL STEARATE (and) PEG-100 STEARATE | 0.80 |
| | ISONONYL ISONONANOATE | 1.00 |
| | PENTAERYTHRITYL TETRAISOSTEARATE | 1.00 |
| | MYRISTYL MYRISTATE | 1.23 |
| | CAPRYLIC/CAPRIC TRIGLYCERIDE | 2.00 |
| | BUTYROSPERMUM PARKII (SHEA) BUTTER | 3.55 |
| | CERA ALBA | 2.73 |
| C | POTASSIUM HYDROXIDE | 0.02 |
| | WATER | 0.25 |
| D | SODIUM POLYACRYLATE | 0.80 |

1. weighting the ingredients for Phase A1 into a container and mixing to obtain a transparent mixture, then stirring at 75° C. under a stirring rate of 500 rpm until a yellowish transparent liquid was obtained;
2. heating the Phase B-pre mix at 85° C., then adding it to the yellowish transparent liquid at 75° C. under a stirring rate of 1500 rpm and mixing for 10 minutes to obtain a white emulsion;
3. adding the phase C premix dropwisely at 75° C. under a stirring rate of 1500 rpm to obtain a white emulsion;
4. adding the phase D premix at 75° C. under a stirring rate of 1500 rpm to obtain a white thin cream, then cooling down to room temperature with stirring to obtain the composition.

### Evaluation

### Lamellar Structure

Whether a lamellar structure is formed was evaluated as follows.

Compositions was observed using a Leica DLMB microscope under 90° cross polarized light, and microscopic pictures were taken.

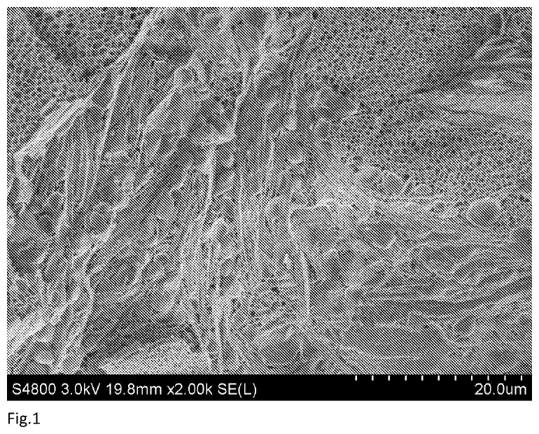

FIG. 1 shows polarized light microscopy image of the composition obtained in invention example 1.

Similar photos were also obtained for the compositions of invention examples 2-5 (IE.2-5).

It can be seen that a lamellar structure is formed in the compositions of invention examples 1-5 (IE.1-5).

Similar photos were not obtained for the compositions of comparative examples 1-4 (CE.1-4).

### Consistency, Spreadability, and Skin Stickness

Consistency was evaluated by 5 sensory experts as follows.

The sensory experts were trained to score within 1-15 based on 15 standard products with the softest consistency to the hardest consistency suitable for skincare creams, wherein 1 means the softest consistency and 15 means the hardest consistency.

### Scoring Standard for Consistency:

5: the consistency is high, the sensory expert gives score >12;
4: the consistency is rather high; the sensory expert gives score 12-10;
3: the consistency is a little bit low, the sensory expert gives score 8-6;
2: the consistency is rather low, the sensory expert gives score 5-4; and
1: the consistency is too low, the sensory expert gives score <4.

Spreadability and skin stickness upon application of each composition were evaluated by 5 skincare formulation experts according to the following steps:
repeatly apply the product to be evaluated 3 times on the same area of the forearm using the same force;
weigh the weight loss of the product;
measure the size of the area on the forearm where the product is applied;
calculate the weight loss per square centimeter.

Finally, scores were given by the experts on the spreadability and skin stickness.

### Scoring Standard for Spreadability:

5: very good;
4: basically good;
3: acceptable;
2: slightly poor and not acceptable;
1: poor, not acceptable.

Scoring standard for skin stickness:
5=not sticky;
3=acceptable stickiness;
1=very sticky.

The results of lamellar-microgel inter-penetration network texture, consistency, spreadability, and skin stickness of each composition prepared above were listed in Table 4.

TABLE 4

| | IE. | IE. | IE. | IE. | IE. | CE. | CE. | CE. | CE. |
|---|---|---|---|---|---|---|---|---|---|
| PROPERTY | 1 | 2 | 3 | 4 | 5 | 1 | 2 | 3 | 4 |
| IPN texture (Y/N) | Y | Y | Y | Y | Y | N | N | N | N |
| Consistency | 4 | 5 | 5 | 4 | 5 | 3 | 4 | 3 | 2 |
| Spreadability | 5 | 5 | 5 | 5 | 5 | 2 | 2 | 2 | 5 |
| Skin stickness | 3 | 3 | 5 | 3 | 3 | 3 | 2 | 1 | 1 |

Y: yes; N: no.

It can be seen from Table 4 that compositions of invention examples 1-5 have a lamellar-microgel inter-penetration network texture, a suitable consistency, and good spreadability, and can deliver a non-sticky skin feeling.

1: A composition in the form of an oil-in-water emulsion for caring for and/or making up keratin materials, comprising:
(i) at least one structuring agent selected from saturated C14-C22 fatty acids;
(ii) at least one hydrophilic gelling agent capable of swelling in water to form a microgel in the form of particles with a particle size of 50-400 μm; and
(iii) at least one surfactant.

2: The composition according to claim 1, wherein the structuring agent is selected from the group consisting of linear and saturated C14-C18 fatty acids.

3: The composition according to claim 1, wherein the structuring agent is present in an amount ranging from 0.5 wt. % to 30 wt. %, preferably from 1 wt. % to 20 wt. %, more preferably from 1.5 wt. % to 10 wt. %, most preferably from 1.5 wt. % to 7 wt. %, relative to the total weight of the composition.

4: The composition according to claim 1, wherein the hydrophilic gelling agent is selected from the group consisting of alkali metal salts of acrylic acid homopolymers, modified starches, and mixtures thereof.

5: The composition according to claim 1, wherein the hydrophilic gelling agent is selected from the group consisting of sodium polyacrylates, potassium polyacrylates, sodium polyacrylate starch, sodium polyacrylate starch, and mixtures thereof.

6: The composition according to claim 1, wherein the hydrophilic gelling agent is present in an amount ranging from 0.1 wt. % to 10 wt. %, relative to the total weight of the composition.

7: The composition according to claim 1, wherein the surfactant is selected from the group consisting of
mono and polyglyceryl esters of a fatty acid or their ethoxylated derivatives;
esters of polyols with fatty acids with a saturated or unsaturated chain;
sorbitol esters of C8-C24 fatty acids and polyoxyalkylenated derivatives thereof;
esters of fatty acids and glucose or alkylglucose or their ethoxylated derivatives;
sucrose esters;
ethers of a sugar and of C8-C24 fatty alcohols
C14-C24 fatty alcohols;
polyoxyethylene alkyl ether carboxylic acids;
polyoxyethylenated fatty alcohol containing from 6 to 12 oxyethylene units;
polyoxyalkylenated derivative of mono glyceryl ester of a fatty acid,
sarcosinates;
fatty acid ester of polyalkylene glycol; and
mixtures thereof.

8: The composition according to claim 1, wherein the surfactant is present in an amount ranging from 0.2 wt. % to 20 wt. %, relative to the total weight of the composition.

9: The composition according to claim 1, wherein the composition further comprises a saponifier selected from sodium hydroxide, potassium hydroxide, magnesium hydroxide, ammonium hydroxide, monoethanolamine, diethanolamine, triethanolamine, tromethamine, N-methylglucamine, arginine, lysine, arginine, and mixtures thereof.

10: The composition according to claim 9, wherein a molar ratio of the saponifier to the structuring agent selected from saturated C14-C22 fatty acids is from 0.1 mol % to 30 mol %.

11: The composition according to claim 1, wherein the composition further comprises a cosmetic active ingredient.

12: The composition according to claim 1, comprising, relative to the total weight of the composition:
(i) from 1.5 wt. % to 7 wt. % of at least one structuring agent selected from linear and saturated C14-C18 fatty acids;
(ii) from 0.3 wt. % to 2 wt. % of at least one hydrophilic gelling agent selected from alkali metal salts of acrylic acid homopolymers, modified starches, and mixtures thereof; and
(iii) from 0.8 wt. % to 12 wt. % of at least one surfactant selected from mono- and polyglyceryl esters of a fatty acid, ethers of a sugar and of C8-C24 fatty alcohols, fatty acid esters of polyalkylene glycol, C14-C24 fatty alcohols, and mixtures thereof.

13: A non-therapeutic method for caring for and/or making up keratin materials, comprising applying the composition according to claim 1 to the keratin materials.
